(19) United States
(12) Patent Application Publication
(10) Pub. No.: US 2015/0025477 A1
(43) Pub. Date: Jan. 22, 2015

(54) SURGICAL SEAL ASSEMBLY INCLUDING CENTERING MECHANISM

(71) Applicant: Covidien LP, Mansfield, MA (US)

(72) Inventor: Christopher Kelly Evans, Meriden, CT (US)

(73) Assignee: Covidien LP, Mansfield, MA (US)

(21) Appl. No.: 14/286,406

(22) Filed: May 23, 2014

Related U.S. Application Data
(60) Provisional application No. 61/856,787, filed on Jul. 22, 2013.

(51) Int. Cl. A61B 17/34; A61B 17/00; A61M 39/06

(52) U.S. Cl. CPC A61B 17/3423; A61M 39/06; A61B 17/00234; A61B 2017/3419; A61M 2039/0626
USPC 604/256

(57) ABSTRACT

A seal assembly for a surgical access assembly includes a housing defining a center, first and second ring members, an inner seal member, and first and second centering mechanisms. The first ring member is disposed within the housing and includes the inner seal member coupled thereto and extending radially inwardly therefrom. The second ring member is disposed within the housing annularly about the first ring member. The inner seal member is configured to sealingly receive a surgical instrument inserted through the housing. The first centering mechanism is configured to bias the first ring member towards a concentric orientation relative to the second ring member. The second centering mechanism is concentrically disposed about the first centering mechanism and is configured to bias the second ring member towards a concentric orientation relative to the center of the housing.

### CROSS-REFERENCE TO RELATED APPLICATIONS

This application claims the benefit of and priority to U.S. Provisional Patent Application No. 61/856,787, filed Jul. 22, 2013, the entire disclosure of which is incorporated by reference herein.

### BACKGROUND

1. Technical Field

The present disclosure relates to surgical access devices and, more particularly, to a surgical seal assembly including a centering mechanism for centering the surgical seal assembly relative to the surgical access device.

2. Background of Related Art

In an effort to reduce trauma and recovery time, many surgical procedures are performed through small openings in the skin such as an incision or a natural body orifice. These procedures, referred to as endoscopic surgical procedures, include laparoscopic procedures, which are generally performed within a patient's abdomen, and thoracic procedures, which are generally performed with a patient's chest cavity. Throughout the present disclosure, the term “minimally invasive” should be understood to encompass any and all such procedures.

During a typical minimally invasive procedure, the clinician creates an opening through the patient's body wall using an obturator or trocar and thereafter or simultaneously therewith positions an access assembly within the opening. The access assembly is configured and dimensioned to receive one or more surgical instruments to facilitate positioning within an internal work site adjacent the tissue that is the subject of the procedure. In some minimally invasive procedures, prior to the introduction of surgical instrumentation into the patient's body, insufflation fluids, e.g., gasses or liquids, are used to enlarge the area surrounding the target surgical site to create a larger and more accessible work area. Accordingly, the maintenance of a substantially fluid-tight seal is desirable during such procedures so as to prevent the escape of the insufflation fluids and the deflation or collapse of the enlarged surgical site.

### SUMMARY

In accordance with the present disclosure, a seal assembly for a surgical access assembly is provided generally including a housing defining a center, first and second ring members, an inner seal member, and first and second centering mechanisms. The first ring member is disposed within the housing. The second ring member is positioned within the housing and is annularly disposed about the first ring member. The inner seal member is coupled to and extends radially inwardly from the first ring member. The inner seal member is configured to sealingly receive a surgical instrument inserted through the housing. The first centering mechanism is coupled between the first and second ring members and is configured to bias the first ring member towards a concentric orientation relative to the second ring member. The second centering mechanism is coupled between the second ring member and the housing and is configured to bias the second ring member towards a concentric orientation relative to the center of the housing.

In embodiments, a peripheral seal member coupled to and extending radially outwardly from the first ring member is provided. The peripheral seal member is configured to sealingly contact an inner surface of the housing to establish a seal between the first ring member and the housing.

In embodiments, the inner seal member is an elastomeric septum seal. The elastomeric septum seal is sealingly engaged about a outer periphery thereof to the first ring member and defines a central opening configured to sealingly receive a surgical instrument inserted through the housing.

In embodiments, the housing is formed from a first housing portion and a second housing portion. The first and second housing portions cooperate to define a chamber configured to retain the first and second ring members, the inner seal member, and the first and second centering mechanisms. The first and second housing portions further cooperate to define a passage extending through the housing that is configured to receive a surgical instrument therethrough.

In embodiments, the first centering mechanism includes a plurality of spokes extending radially outwardly from the first ring member. Free ends of the spokes are configured to contact the second ring member to bias the first ring member towards a concentric orientation relative to the second ring member. Alternatively, the first centering mechanism may include an annular bellows member extending radially outwardly from the first ring member. An outer periphery of the annular bellows member is configured to contact the second ring member to bias the first ring member towards a concentric orientation relative to the second ring member.

In embodiments, the first and second centering mechanisms define different configurations and/or different biasing forces.

In embodiments, the second centering mechanism includes a plurality of spokes extending radially outwardly from the second ring member. Free ends of the spokes are configured to contact the housing to bias the second ring member towards a concentric orientation relative to the center of the housing.

In embodiments, the first and second centering mechanisms are fixedly engaged to the first and second ring members, respectively. In such embodiments, the first and second centering mechanisms including the respective first and second ring members may be removably coupled to one another and to the housing.

In embodiments, the seal assembly further includes a cannula assembly having a base member and an elongated tubular member extending distally from the base member. The housing is releasably engagable with the base member of the cannula assembly.

In embodiments, the seal assembly further includes a zero closure seal.

Also provided in accordance with the present disclosure is a method of surgery including inserting a surgical access assembly including a seal assembly, e.g., according to any of the above-described embodiments, into tissue and inserting a surgical instrument through the housing and the inner seal member of the seal assembly such that the inner seal member sealingly engages the surgical instrument, the first centering mechanism biases the inner seal member towards a concentric orientation relative to the second ring member, and the second centering mechanisms biases the second ring member towards a concentric orientation relative to the housing.

In embodiments, prior to inserting the surgical access assembly into tissue, a desired seal assembly is selected and engaged to the surgical access assembly. Selecting a desired seal assembly may include selecting a desired first centering mechanism, selecting a desired second centering mechanism, coupling the first and second centering mechanisms to one another, and positioning the first and second centering mechanisms within the housing.

In embodiments, the method further includes insufflating the internal surgical site prior to insertion of the surgical instrument. The sealing engagement of the inner seal member about the surgical instrument maintains the internal surgical site in an insufflated state upon insertion of the surgical instrument into the internal surgical site.

In embodiments, a zero closure valve is disposed within the surgical access assembly to maintain the internal surgical site in an insufflated state in the absence of the surgical instrument inserted therethrough.

In embodiments, inserting the surgical access assembly through tissue includes positioning a obturator assembly within the surgical access assembly and advancing the surgical access assembly through tissue such that a distal tip of the obturator assembly dissects tissue.

### BRIEF DESCRIPTION OF THE DRAWINGS

Various aspects and features of the present disclosure are described herein with reference to the drawings wherein:

FIG. 1 is a perspective view from the proximal end of a surgical access assembly provided in accordance with the present disclosure;

FIG. 2 is a side view of an obturator assembly of the surgical access assembly of FIG. 1;

FIG. 3 is a side view of a cannula assembly of the surgical access assembly of FIG. 1 shown with a housing of the cannula assembly separated therefrom;

FIG. 4 is an exploded, perspective view of the surgical access assembly of FIG. 1;

FIG. 5 is an exploded, perspective view of the housing of the cannula assembly of FIG. 3;

FIG. 6 is an exploded, perspective view of an embodiment of an insert seal assembly configured for use with the cannula assembly of FIG. 3;

FIG. 7 is a top view of the insert seal assembly of FIG. 6;

FIG. 8 is a side, cross-sectional view of the insert seal assembly of FIG. 6;

FIG. 9 is an exploded, perspective view of another embodiment of an insert seal assembly configured for use with the cannula assembly of FIG. 3;

FIG. 10 is a top view of the insert seal assembly of FIG. 9; and

FIG. 11 is a side, cross-sectional view of the insert seal assembly of FIG. 9.

### DETAILED DESCRIPTION

Particular embodiments of the present disclosure are described in detail hereinbelow with reference to the accompanying drawings wherein like reference numerals identify similar or identical structural elements. As shown in the drawings and described throughout the following description, as is traditional when referring to relative positioning on a surgical instrument, the term “proximal” refers to the end of the apparatus which is closer to the user and the term “distal” refers to the end of the apparatus which is further away from the user. Well-known functions or constructions are not described in detail to avoid obscuring the present disclosure in unnecessary detail.

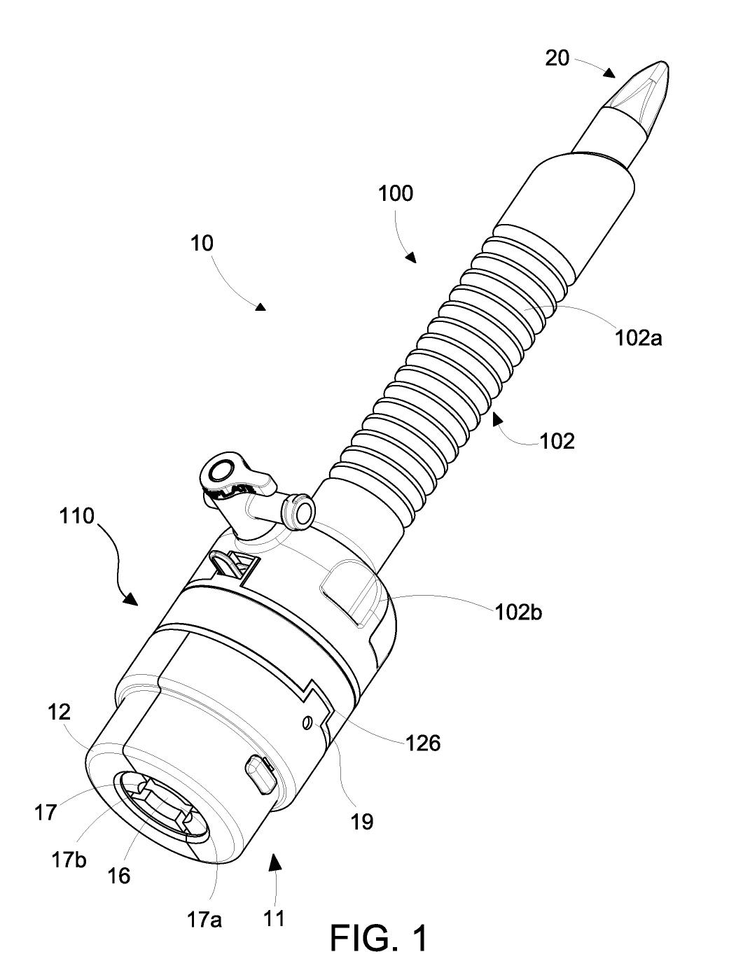

Turning now to FIGS. 1-4, a surgical access assembly provided in accordance with the present disclosure is shown generally identified by reference numeral 10. Surgical access assembly 10 is configured to provide a substantially fluid-tight seal between an internal body cavity of a patient and the outside atmosphere before, during, and after insertion of one or more surgical instruments through surgical access assembly 10 and into the internal body cavity. Surgical access assembly 10 includes an obturator assembly 11, and a cannula assembly 100 that is configured to at least partially receive obturator assembly 11.

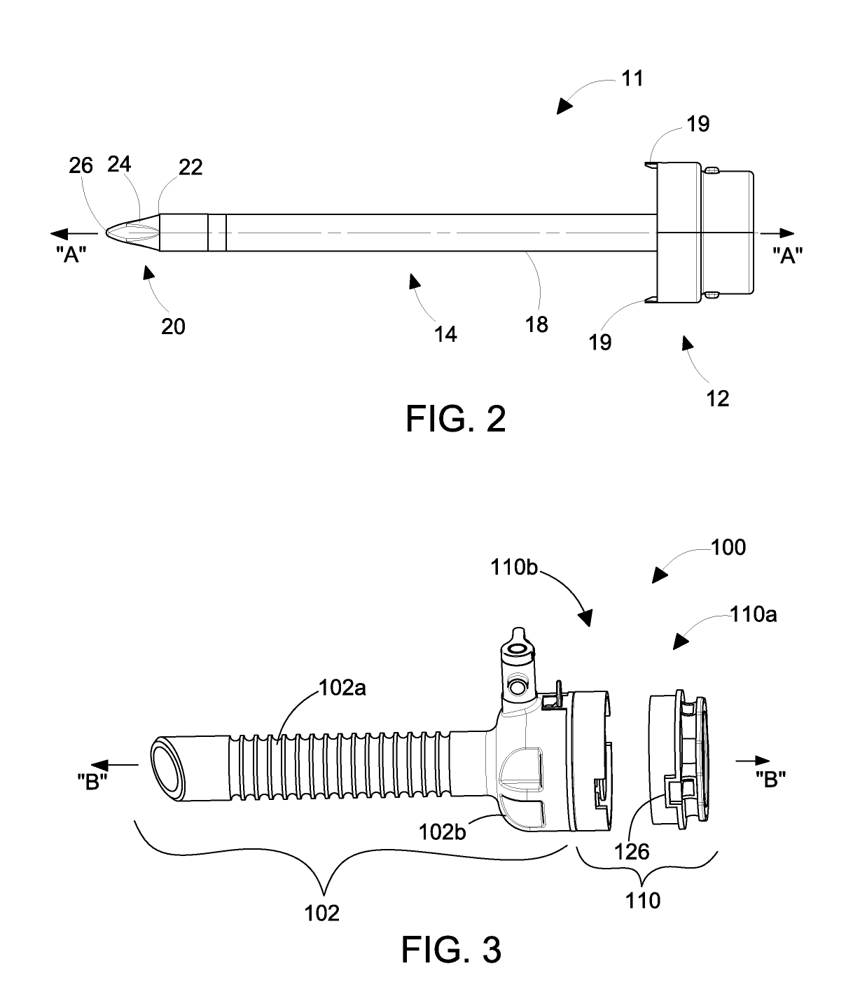

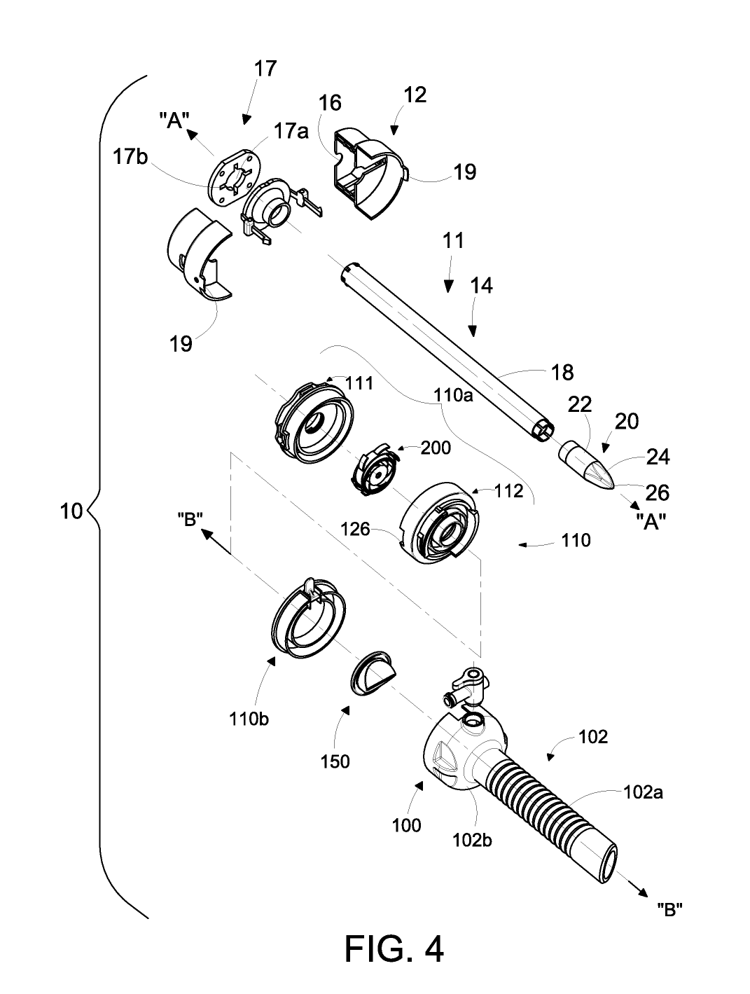

With particular reference to FIGS. 2 and 4, obturator assembly 11 defines a longitudinal axis “A-A” and includes an obturator housing 12 disposed in mechanical cooperation with an elongated obturator member 14. Obturator housing 12 of obturator assembly 11 defines an opening 16 and includes a scope retention member 17 adjacent opening 16. Scope retention member 17 is fabricated from an elastomeric material, and defines a central opening 17a for receiving the endoscope (not shown) and four radial slits 17b extending outwardly from central opening 17a that permit flexure of scope retention member 17. Scope retention member 17 is configured to engage an outer surface of an endoscope (not shown) inserted therethrough in frictional engagement therewith to assist in retaining the relative positioning of the endoscope (not shown) within the obturator assembly 11.

Elongated obturator member 14, as mentioned above, is configured for insertion through cannula assembly 100 (FIG. 3). Elongated obturator member 14 extends distally from obturator housing 12 and includes an obturator shaft 18 mechanically coupled to obturator housing 12, and an optical member 20 disposed at the distal end of obturator shaft 18. Obturator shaft 18 may be made from steel, a polymeric material, or any other suitable material. Optical member 20 defines a hollow interior and includes a proximal section 22, a central section 24, and an atraumatic guiding nub 26. Atraumatic guiding nub 26 permits initial insertion within an opening, e.g., a pre-cut scalpel incision, in the tissue and facilitates the advancement of the optical member 20 between the tissue layers to gently dissect tissue, without cutting or incising tissue. After initial insertion and continued distal insertion, the central section 24 and the proximal portion 22 continue to gently enlarge the opening in tissue. A distal viewing tip of an endoscope (not shown) is insertable through obturator shaft 18 and into the hollow interior of optical member 20 to facilitate visualization of tissue adjacent optical member 20 during insertion and advancement through tissue.

Referring to FIGS. 3 and 4, cannula assembly 100 of surgical access assembly 10 (FIG. 1) includes an elongated portion 102 defining a longitudinal axis “B-B,” and a housing 110 including a proximal housing component 110a and a distal housing component 110b. Elongated portion 102 includes an elongated tubular member 102a and a base member 102b. Base member 102b is configured to releasably engage housing 110. Elongated tubular member 102a extends distally from base member 102b and may be formed from a translucent material, although other configurations are also contemplated. As will be described in greater detail below, proximal housing component 110a of housing 110 is configured to enclose an insert seal assembly 200 therein. Distal housing component 110b of housing 110 is configured to enclose a zero-closure seal 150 therein that maintains a substantially fluid-tight seal between an internal body cavity of a patient and the outside atmosphere in the absence of surgical instrumentation inserted through surgical access assembly 10. It is contemplated that cannula assembly 100 be configured to sealingly accept surgical instrumentation of varying diameters, e.g., from about 5 mm to about 15 mm. Proximal housing component 110a further includes a pair of notches 126 configured to mechanically engaged a pair of corresponding latches 19 disposed on obturator housing 12 to selectively lock and unlock obturator assembly 11 to and from cannula assembly 100. Other suitable releasable engagement structures are also contemplated.

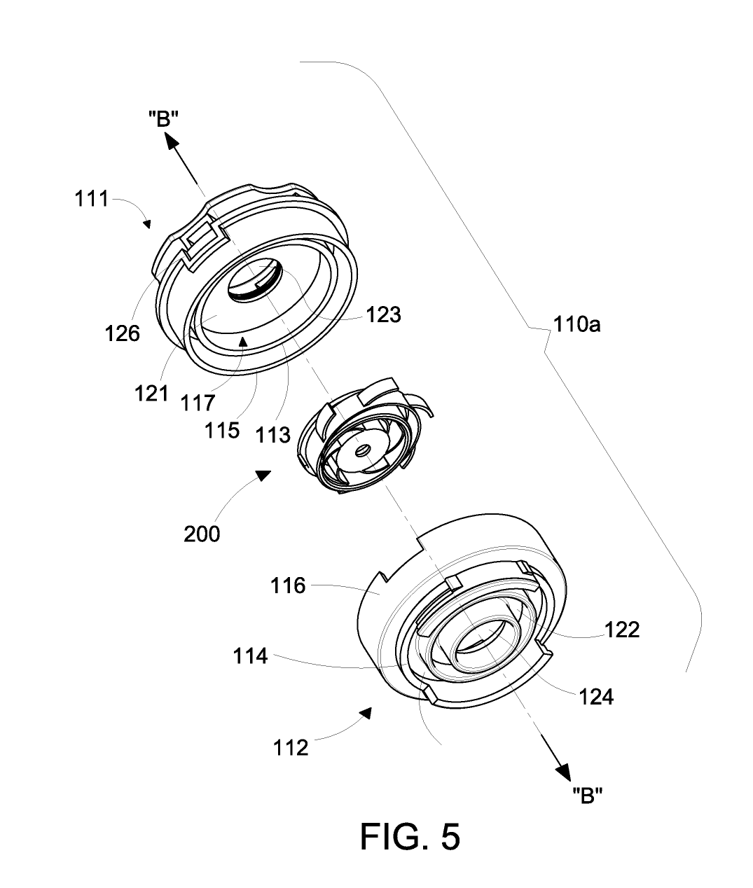

With reference to FIG. 5, proximal housing component 110a of housing 110 includes first and second portions 111, 112, respectively, that are selectively engagable with each other via snap-fit engagement or other suitable arrangement to form proximal housing component 110a and retain insert seal assembly 200 therebetween. First portion 111 of proximal housing component 110a includes an inner annular member 113 and an outer annular member 115 disposed radially outwardly of inner annular member 113. Inner annular member 113 defines an inner wall 117 and is configured to at least partially receive insert seal assembly 200 within the area defined by inner wall 117. A disc member 121 disposed within inner annular member 113 defines a central passage 123 having a reduced diameter as compared to the area defined within inner wall 117.

Second portion 112 of proximal housing component 110a likewise includes an inner annular member 114 and an outer annular member 116 disposed radially outwardly of inner annular member 114. A disc member 122 disposed within inner annular member 114 defines a central passage 124 having a reduced diameter as compared to the area defined within inner annular member 114. Outer annular members 115, 116 of first and second portions 111, 112, respectively, are selectively engagable with each other to releasably secure first and second potions 111, 112 to one another to form proximal housing component 110a. Upon engagement of first and second portions 111, 112 to form proximal housing component 110a, inner annular members 113, 114 and discs 121, 122 of first and second portions 111, 112, respectively, cooperate to form a chamber configured to receive and retain insert seal assembly 200 therein. More specifically, insert seal assembly 200 is retained axially within the chamber between discs 121, 122 and radially within the chamber via inner wall 117 of inner annular member 113 of first portion 111 and/or the inner wall (not shown) of inner annular member 114 of second portion 112. Insert seal assembly 200 is described in detail below.

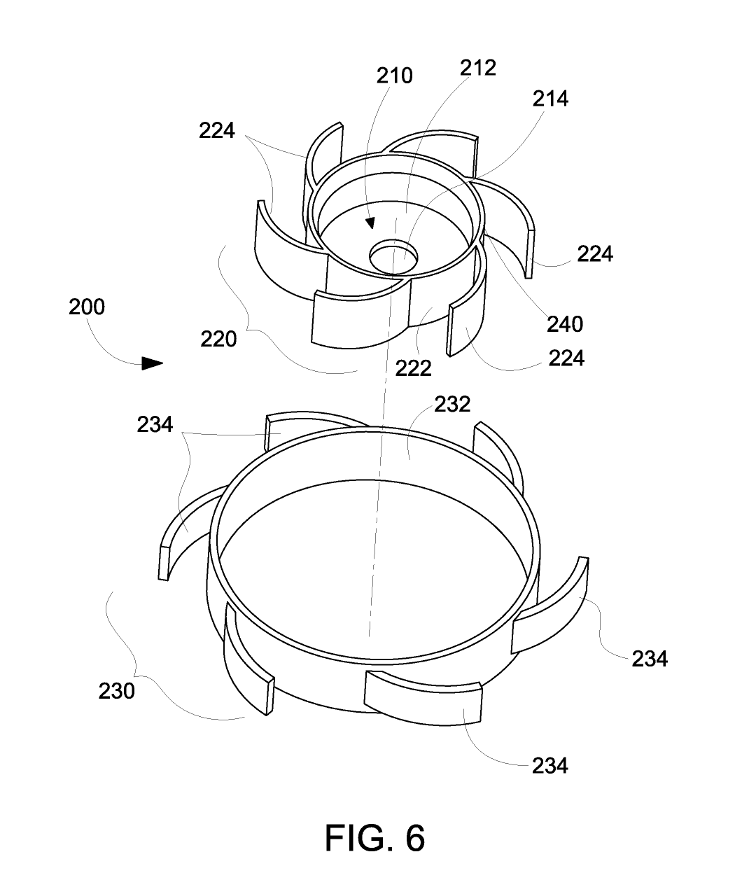

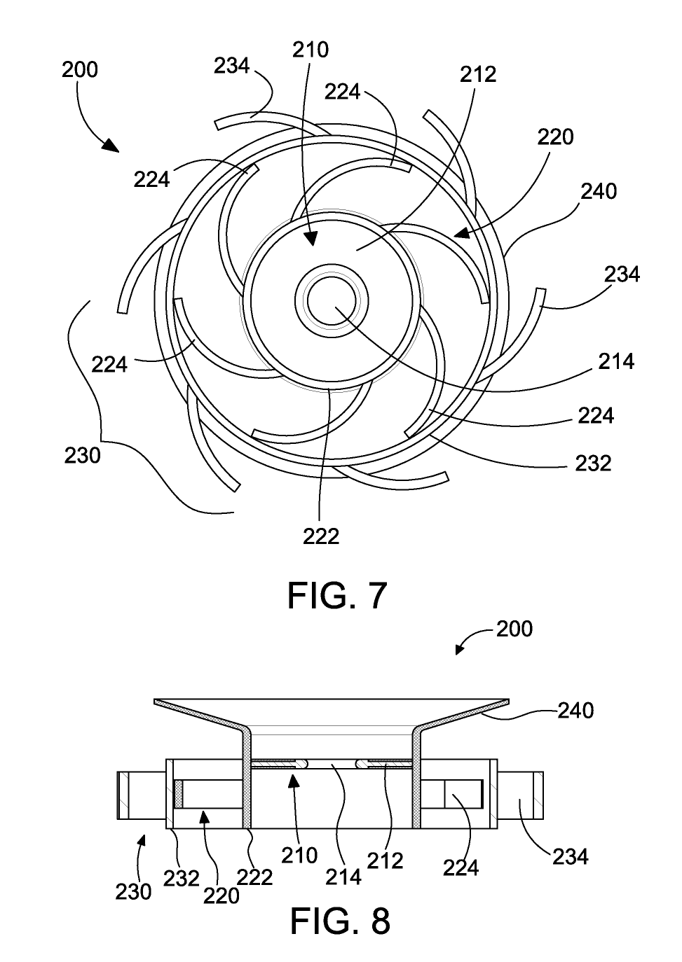

Turning now to FIGS. 6-8, in conjunction with FIG. 5, an embodiment of an insert seal assembly 200 is shown generally including an elastomeric septum seal 210, first and second centering mechanisms 220, 230 including first and second ring members 222, 232, respectively, and a peripheral seal 240 (peripheral seal 240 has been removed from FIGS. 4 and 5, and a portion of peripheral seal 240 has been removed from FIG. 6 to better illustrate the other components of insert seal assembly 200). Septum seal 210 is configured provide a substantially fluid-tight seal about the outer surface of an instrument passing therethrough and includes a flat seal portion 212 having an orifice 214 at or near its radial center. Septum seal 210 is sealing engaged about its outer periphery to first ring member 222, e.g., via adhesion, snap-fit retention, or other suitable sealing engagement. Peripheral seal 240 is configured to provide a substantially fluid-tight seal between insert seal assembly 200 and housing 110 (FIG. 5), e.g., inner wall 117 of inner annular member 113 of first portion 111 of proximal housing component 110a and/or the inner wall (not shown) of inner annular member 114 of second portion 112 of proximal housing component 110a (see FIG. 5). Peripheral seal 240 extends radially outwardly from first ring member 222 and is sealingly engaged to first ring member 222, e.g., via adhesion, snap-fit retention, or other suitable sealing engagement.

Referring again to FIGS. 6-8, first centering mechanism 220 is positioned between first and second ring members 222, 232, respectively, while second centering mechanism 230 extends radially outwardly from second ring member 232, e.g., for positioning between insert seal assembly 200 and proximal housing component 110a (FIG. 5), thus allowing first and second ring members 222, 232 to “float” relative to one another and to proximal housing component 110a. As can be appreciated, as a result of this configuration, first and second centering mechanisms 220, 230 are concentrically disposed relative to one another and to septum seal 210. It is contemplated that additional centering mechanisms and ring members may be provided, in concentric relation relative to the other centering mechanisms as well as to septum seal 210. Further, as second centering mechanism 230 is positioned within insert seal assembly 200 and retained therein only under bias, and as first centering mechanism 220 is positioned within second centering mechanism 230 and retained therein only under bias, various differently configured centering mechanisms may be interchangeably provided for use depending on a particular purpose, e.g., the diameter of instrumentation to be inserted through surgical access assembly 10 (FIG. 1), the required degree of manipulation of the instrumentation, the procedure to be performed, a patient's anatomy, etc. Alternatively, proximal housing component 110a (FIG. 5) may be provided as a partially or fully integrated component such that the entire proximal housing component 110a (FIG. 5) or only the insert seal assembly 200 thereof is replaceable to provide a seal assembly of a desired configuration.

Continuing with reference to FIGS. 6-8 first centering mechanism 220 includes a plurality of spring elements or spokes 224 equally spaced about and extending radially outwardly from first ring member 222. Spokes 224 define a pinwheel configuration about the outer periphery of first ring member 222 and generally trend in a clockwise direction, although other configurations are also contemplated. Spokes 224 are configured to contact an inner surface of second ring member 232 towards the free ends of spokes 224 to bias first ring member 222 and, thus, septum seal 210 at or near the radial center of second ring member 232 and to inhibit lateral movement of septum seal 210 within second ring member 232.

Second centering mechanism 230, similar to first centering mechanism 220, includes a plurality of spring elements or spokes 234. Spokes 234 are equally spaced about and extend radially outwardly from second ring member 232. Spokes 234 define a pinwheel configuration about the outer periphery of second ring member 232 and generally trend in a counterclockwise direction, although other configurations are also contemplated, e.g., this configuration may be reversed (wherein spokes 224 trend in a counterclockwise direction and spokes 234 trend in a clockwise direction) or both sets of spokes 224, 234 may trend in a similar direction. Spokes 234 are configured to contact inner wall 117 of inner annular member 113 of first portion 111 of proximal housing component 110a and/or the inner wall (not shown) of inner annular member 114 of second portion 112 of proximal housing component 110a (see FIG. 5) to bias insert seal assembly 200 at or near the radial center of proximal housing component 110a (FIG. 5) and to inhibit lateral movement of insert seal assembly 200 within proximal housing component 110a (FIG. 5).

As mentioned above, second centering mechanism 230 provides sufficient bias so as to maintain insert seal assembly 200 at or near the radial center of proximal housing component 110a (FIG. 5), while first centering mechanism 220 provides sufficient bias so as to maintain septum seal 210 at or near the radial center of second ring member 232. As a result of this configuration, the requirements of both maintaining alignment of insert seal assembly 200 and inhibiting distortion and/or leaking of septum seal 210, e.g., separation of septum seal 210 from an instrument disposed therethrough, are achieved.

Spokes 224, 234 of first and second centering mechanisms 220, 230, respectively, may be configured to trend in similar directions or in opposite directions, as mentioned above. In some embodiments, the orientation of either first or second centering mechanism 220, 230 may be inverted prior to insertion of first centering mechanism 220 into second centering mechanism 230, e.g., within second ring member 232, to achieve a desired trend of spokes 224, 234 relative to one another. Further, the material forming spokes 224, 234, the thickness and/or length of spokes 224, 234, etc. may be chosen to achieve a centering mechanism 220, 230 having a desired configuration and/or spring constant and, thus, a desired biasing force. As mentioned above, various differently configured and interchangeable centering mechanisms may be provided and combined in any suitable combination to achieve a desired effect. That is, a particular first centering mechanism having a desired configuration and/or spring constant may be used in conjunction with a particular second centering mechanism having a desired configuration and/or spring constant to achieve a desired insert seal assembly.

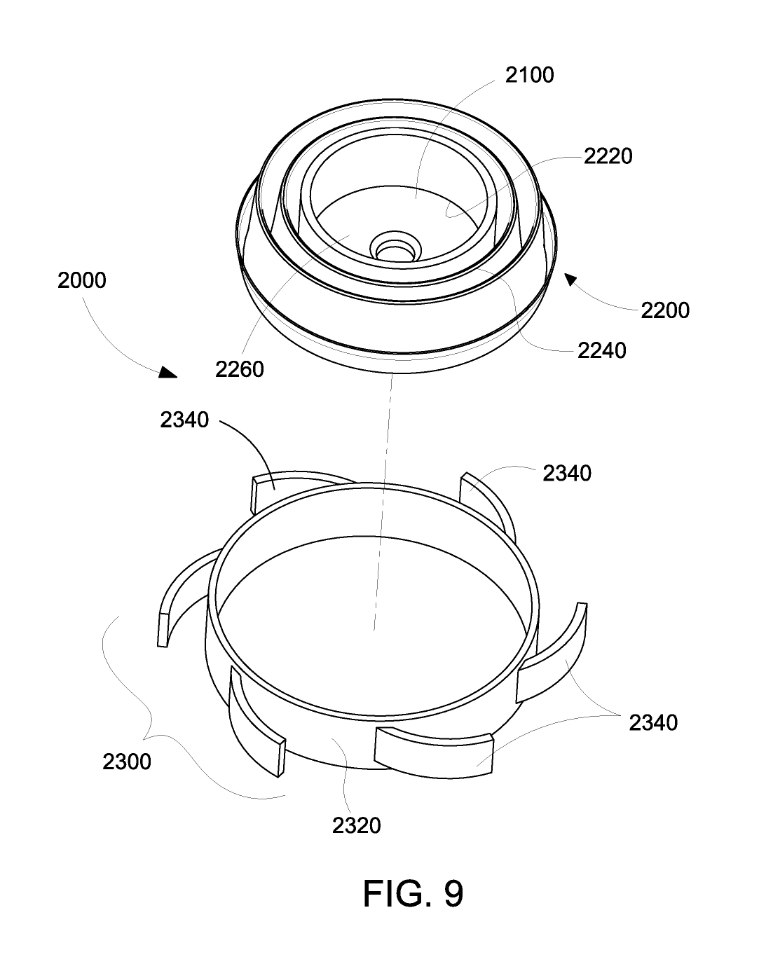

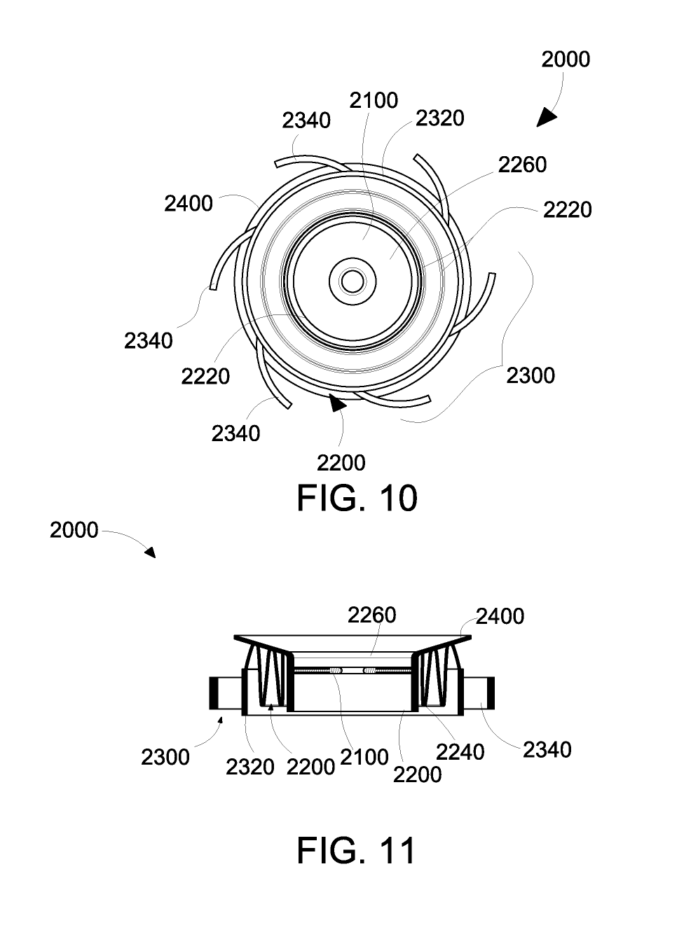

Turning now to FIGS. 9-11, another embodiment of an insert seal assembly 2000 is shown generally including an elastomeric septum seal 2100, first and second centering mechanisms 2200, 2300 including first and second ring members 2220, 2320, respectively, and a peripheral seal 2400 (peripheral seal 2400 has been removed from FIG. 9 to better illustrate the other components of insert seal assembly 2000). Insert seal assembly 2000 is similar to insert seal assembly 200 (FIGS. 5-8) detailed above and, thus, only the differences between insert seal assembly 2000 and insert seal assembly 200 (FIGS. 5-8) will be described in detail hereinbelow, while similarities between insert seal assembly 200 (FIGS. 5-8) and insert seal assembly 2000 will only be summarily described or omitted entirely.

Septum seal 2100 is disposed within first ring member 2220 and is configured provide a substantially fluid-tight seal about the outer surface of an instrument passing therethrough. Peripheral seal 2400 extends radially outwardly from first ring member 2220 and is configured to provide a substantially fluid-tight seal between insert seal assembly 2000 and proximal housing component 110a (FIGS. 4-5).

First centering mechanism 2200 is positioned between first and second ring members 2220, 2320, respectively, while second centering mechanism 2300 extends radially outwardly from second ring member 2320. First centering mechanism 2200 includes an annular bellows member 2240 defining a central opening 2260 configured to receive first ring member 2220. Annular bellows member 2240 may be engaged to or biased against first ring member 2220 about its inner annular surface and engaged to or biased against second ring member 2320 about its outer annular surface. Annular bellows member 2240 is resiliently expandable and contractable from its at-rest position and defines a generally sine-wave shaped cross-sectional configuration (see FIG. 11), e.g., wherein the frequency of the “sine-wave” is varied upon expansion and contraction. Annular bellows member 2240 may be formed from an elastomeric or other suitable resilient material for biasing first ring member 2220 and, thus, septum seal 2100 at or near the radial center of second ring member 2320, whereby first ring member 2220 “floats” relative to second ring member 2320 and proximal housing component 110a (FIGS. 4-5).

Second centering mechanism 2300 is similar to second centering mechanism 230 of insert seal assembly 200 (FIGS. 5-8), e.g., second centering mechanism 2300 includes a plurality of biasing spokes 2340 extending from second ring member 2320. However, it is also envisioned that the configuration of insert seal assembly 2000 be reversed, e.g., wherein first centering mechanism 2200 includes a plurality of spokes while second centering mechanism 2300 includes an annular bellows member. Alternatively, both centering mechanisms 2200, 2300 may include an annular bellows member.

Insert seal assembly 2000 provides the same advantages described above with respect to insert seal assembly 200 (FIGS. 5-8) and may include any of the features thereof. In particular, as mentioned above, various differently configured centering mechanisms may be interchangeably provided for use depending on a particular purpose to achieve a desired configuration. With respect to centering mechanisms including an annular bellows member, various different configurations and/or spring constants may be achieved by varying the thickness, length, material, at-rest amplitude and/or at-rest frequency of the bellows member, etc. Thus, similar or different types of centering mechanisms, e.g., spoked, bellowed, etc., having a particular configuration and/or spring constant may be selected and combined with one another to achieve a desired insert seal assembly.

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A seal assembly for a surgical access assembly, comprising:
a housing defining a center;
a first ring member disposed within the housing;
a second ring member disposed within the housing, the second ring member annularly disposed about the first ring member;
an inner seal member coupled to and extending radially inwardly from the first ring member, the inner seal member configured to sealingly receive a surgical instrument inserted through the housing;
a first centering mechanism coupled between the first and second ring members, the first centering mechanism configured to bias the first ring member towards a concentric orientation relative to the second ring member; and
a second centering mechanism coupled between the second ring member and the housing, the second centering mechanism configured to bias the second ring member towards a concentric orientation relative to the center of the housing.

2. The seal assembly according to claim 1, further comprising a peripheral seal member coupled to and extending radially outwardly from the first ring member, the peripheral seal member configured to sealingly contact an inner surface of the housing to establish a seal between the first ring member and the housing.

3. The seal assembly according to claim 1, wherein the inner seal member is an elastomeric septum seal that is sealingly engaged about a outer periphery thereof to the first ring member and defines a central opening configured to sealingly receive a surgical instrument inserted through the housing.

4. The seal assembly according to claim 1, wherein the housing is formed from a first housing portion and a second housing portion, the first and second housing portions cooperating to define a chamber configured to retain the first and second ring members, the inner seal member, and the first and second centering mechanisms, the first and second housing portions cooperating to define a passage extending through the housing that is configured to receive a surgical instrument therethrough.

5. The seal assembly according to claim 1, wherein the first centering mechanism includes a plurality of spokes extending radially outwardly from the first ring member, wherein free ends of the spokes are configured to contact the second ring member to bias the first ring member towards a concentric orientation relative to the second ring member.

6. The seal assembly according to claim 1, wherein the first centering mechanism includes an annular bellows member extending radially outwardly from the first ring member, wherein an outer periphery of the annular bellows member is configured to contact the second ring member to bias the first ring member towards a concentric orientation relative to the second ring member.

7. The seal assembly according to claim 1, wherein the first and second centering mechanisms define at least one of different configurations and different biasing forces.

8. The seal assembly according to claim 1, wherein the second centering mechanism includes a plurality of spokes extending radially outwardly from the second ring member, free ends of the spokes configured to contact the housing to bias the second ring member towards a concentric orientation relative to the center of the housing.

9. The seal assembly according to claim 1, wherein the first and second centering mechanisms are fixedly engaged to the first and second ring members, respectively.

10. The seal assembly according to claim 9, wherein the first and second centering mechanisms including the respective first and second ring members are removably coupled to one another and to the housing.

11. The seal assembly according to claim 1, further comprising a cannula assembly including a base member and an elongated tubular member extending distally from the base member, the housing releasably engagable with the base member of the cannula assembly.

12. The seal assembly according to claim 1, further comprising a zero closure valve.

13. A method of surgery, comprising:
inserting a surgical access assembly into tissue, the surgical access assembly including a seal assembly including:
a housing;
a first centering mechanism including a first ring member disposed within the housing, the first ring member coupled to the inner seal member;
an inner seal member disposed within the housing, the inner seal member coupled to and positioned within the first ring member;
a second centering mechanism including a second ring member disposed within the housing, the second centering mechanism annularly disposed about the first centering mechanism; and
inserting a surgical instrument through the housing and the inner seal member and into an internal surgical site such that the inner seal member sealingly engages the surgical instrument, the first centering mechanism biases the inner seal member towards a concentric orientation relative to the second ring member, and the second centering mechanism biases the second ring member towards a concentric orientation relative to the housing.

14. The method according to claim 13, wherein, prior to inserting the surgical access assembly into tissue, the method includes:
selecting a desired seal assembly; and
engaging the selected seal assembly to the surgical access assembly.

15. The method according to claim 14, wherein selecting a desired seal assembly includes:
selecting a desired first centering mechanism;
selecting a desired second centering mechanism;
coupling the first and second centering mechanisms to one another; and
positioning the first and second centering mechanisms within the housing.

16. The method according to claim 13, further comprising insufflating the internal surgical site prior to insertion of the surgical instrument, wherein the sealing engagement of the inner seal member about the surgical instrument maintains the internal surgical site in an insufflated state.

17. The method according to claim 16, further comprising a zero closure valve disposed within the surgical access assembly, the zero closure valve maintaining the internal surgical site in an insufflated state in the absence of the surgical instrument inserted therethrough.

18. The method according to claim 13, wherein inserting the surgical access assembly into tissue includes positioning a obturator assembly within the surgical access assembly and advancing the surgical access assembly through tissue such that a distal tip of the obturator assembly dissects tissue.

19. The method according to claim 13, wherein the first centering mechanism includes a plurality of spokes extending radially outwardly from the first ring member, wherein free ends of the spokes are configured to contact the second ring member to bias the first ring member towards a concentric orientation relative to the second ring member.

20. The method according to claim 13, wherein the first centering mechanism includes an annular bellows member extending radially outwardly from the first ring member, wherein an outer periphery of the annular bellows member is configured to contact the second ring member to bias the first ring member towards a concentric orientation relative to the second ring member.
